(19) United States
(12) Patent Application Publication
(10) Pub. No.: US 2021/0007614 A1
(43) Pub. Date: Jan. 14, 2021

(54) BIOLOGICAL INFORMATION MEASUREMENT APPARATUS, METHOD, AND PROGRAM

(71) Applicants: OMRON Corporation, Kyoto-shi, Kyoto (JP); OMRON HEALTHCARE CO., LTD., Muko-shi, Kyoto (JP)

(72) Inventors: Satoshi YASE, Nara-shi (JP); Keigo KAMADA, Tokyo (JP); Hisashi OZAWA, Kyoto-shi (JP); Ayaka IWADE, Nara-shi (JP); Masayuki SUGANO, Uji-shi (JP); Keisuke SAITO, Suita-shi (JP); Yasuhiro KAWABATA, Kyoto (JP)

(73) Assignees: OMRON Corporation, Kyoto-shi, Kyoto (JP); OMRON HEALTHCARE CO., LTD., Muko-shi, Kyoto (JP)

(21) Appl. No.: 17/041,160

(22) PCT Filed: Apr. 2, 2019
(86) PCT No.: PCT/JP2019/014625

§ 371 Date: Sep. 24, 2020

(30) Foreign Application Priority Data
Apr. 12, 2018 (JP) 2018-077082

(51) Int. Cl. A61B 5/021; A61B 5/00

(52) U.S. Cl. CPC A61B 5/02116; A61B 5/0015; A61B 2562/0219; A61B 5/02125; A61B 5/0006; A61B 5/7246

(57) ABSTRACT

Provided is a technique for detecting a state of an occurrence of a body motion of a measurement target person affecting measurement by using an apparatus configured to measure biological information using a radio wave. A biological information measurement apparatus (1) according to an aspect of the present disclosure includes: a transmitter (3) configured to transmit a radio wave to a measurement site of a living body; a receiver (4) configured to receive a reflected wave of the radio wave by the measurement site and output a waveform signal of the reflected wave; a feature extractor (1051) configured to extract information indicating a feature of a waveform from the waveform signal; and a body motion detector (1052) configured to detect a state of an occurrence of a body motion of the living body affecting measurement of the biological information, based on the extracted information indicating the feature of the waveform.

### FIELD

The present invention relates to a biological information measurement apparatus, method, and program for measuring biological information using, for example, a radio wave.

### BACKGROUND

As an apparatus for measuring biological information using a radio wave, there has been known an apparatus that includes a transmission antenna and a reception antenna arranged to face a measurement site, transmits a radio wave (measurement signal) from the transmission antenna toward the measurement site (target object), and receives a reflected wave (reflected signal) of the transmitted radio wave by the measurement site, to measure biological information (see, for example, Patent Literature 1).

### CITATION LIST

### Patent Literature

### PATENT LITERATURE 1: Japanese Patent No. 5879407

### SUMMARY

### Technical Problem

In the case of measurement, for example, the measurement site is generally a pulse wave (or a signal related to a pulse wave) as biological information, a wrist or an upper arm. In the case of performing measurement by wearing a wearable device on a wrist, the adoption of a configuration in which a transmission antenna and a reception antenna (collectively referred to as a “transmission-reception antenna pair”, as appropriate) are installed in a wrist-wearing strap of the device, so that a pulse wave signal is measured by the transmission-reception antenna pair, is assumed. In this configuration, the measurement of the biological information is greatly affected by a body motion, making it impossible to properly measure the biological information when a measurement target person (also referred to as a “user”) is moving his or her body. The inventors of the present invention have proposed an apparatus having a function of detecting a body motion together with biological information. However, since this type of apparatus uses a motion sensor such as an acceleration sensor to detect a body motion, the apparatus becomes large, complicated, and expensive.

To resolve the above drawback, the present invention, in one aspect, provides a biological information measurement apparatus, method, and program that allow for detection of a body motion of a user without the addition of another sensor device.

### Solution to Problem

To resolve the above drawback, a biological information measurement apparatus according to a first aspect of the present invention includes: a transmitter configured to transmit a radio wave to a measurement site of a living body; a receiver configured to receive a reflected wave of the radio wave by the measurement site and output a waveform signal of the reflected wave; a feature extractor configured to extract information indicating a feature of a waveform from the waveform signal; and a body motion detector configured to detect a state of an occurrence of a body motion of the living body, affecting measurement of the biological information, based on the extracted information indicating the feature of the waveform.

According to the first aspect of the present invention, information indicating a feature of a waveform is extracted from a waveform signal obtained by transmitting and receiving a radio wave to and from a measurement site, and a state of an occurrence of a body motion of the living body affecting measurement of biological information is detected based on the extracted information indicating the feature of the waveform. Therefore, it is possible to detect a body motion of a user by using the existing configuration included in the biological information measurement apparatus without adding another sensing device such as an acceleration sensor. As a result, the apparatus can be rendered simple, compact, and inexpensive.

A second aspect of the present invention is the biological information measurement apparatus according to the first aspect, wherein the feature extractor is configured to extract information relating to an amplitude of the waveform signal as a feature of the waveform of the waveform signal, and the body motion detector is configured to determine that the body motion has occurred when an amplitude value of the waveform signal exceeds a preset first amplitude value for a time period longer than a preset first duration, based on the extracted information relating to the amplitude of the waveform.

A third aspect of the present invention is the biological information measurement apparatus according to the first aspect, wherein the feature extractor is configured to extract information relating to an amplitude of the waveform signal as a feature of the waveform of the waveform signal, and the body motion detector is configured to determine that the body motion has occurred when an amplitude value of the waveform signal is below a preset first amplitude value for a time period shorter than a preset first duration, based on the extracted information relating to the amplitude of the waveform.

A fourth aspect of the present invention is the biological information measurement apparatus according to the first aspect, wherein the feature extractor is configured to extract information relating to an amplitude of the waveform signal as a feature of the waveform of the waveform signal, and the body motion detector is configured to determine that the body motion has occurred when an amplitude value of the waveform signal exceeds a preset first amplitude value for a time period shorter than a preset first duration, based on the extracted information relating to the amplitude of the waveform.

A fifth aspect of the present invention is the biological information measurement apparatus according to the first aspect, wherein the feature extractor is configured to extract information relating to an amplitude of the waveform signal as a feature of the waveform of the waveform signal, and the body motion detector is configured to determine that the body motion has occurred when an amplitude value of the waveform signal is below a preset first amplitude value for a time period longer than a preset first duration, based on the extracted information relating to the amplitude of the waveform.

According to the second to fifth aspects of the present invention, the amplitude value of the waveform is extracted as a feature of the waveform signal, and the occurrence of the body motion is determined based on a duration of the variation of the amplitude value. Therefore, the occurrence of the body motion can be accurately determined by focusing on both the amplitude of the waveform signal and the duration thereof.

A sixth aspect of the present invention is the biological information measurement apparatus according to the first aspect, wherein the feature extractor is configured to extract information relating to a repetition cycle of the waveform signal as a feature of the waveform of the waveform signal, and the body motion detector is configured to determine that the body motion has occurred when the repetition cycle of the waveform signal exceeds a preset time range, based on the extracted information relating to the repetition cycle of the waveform.

According to the sixth aspect of the present invention, the repetition cycle of the waveform is extracted as a feature of the waveform signal, and the occurrence of the body motion is determined based on the variation of the repetition cycle. Therefore, the body motion can be determined through a relatively simple process, merely by monitoring the change in the repetition cycle of the waveform signal.

A seventh aspect of the present invention is the biological information measurement apparatus according to the first aspect, wherein the feature extractor is configured to extract information relating to an amplitude of the waveform signal as a feature of the waveform of the waveform signal, and the body motion detector is configured to determine that the body motion has occurred when an amplitude value of the waveform signal exceeds a preset first amplitude range, based on the extracted information relating to the amplitude of the waveform.

An eighth aspect of the present invention is the biological information measurement apparatus according to the first aspect, wherein the feature extractor is configured to extract information relating to an amplitude of the waveform signal as a feature of the waveform of the waveform signal, and the body motion detector is configured to determine that the body motion has occurred when an amplitude value of the waveform signal does not exceed a preset second amplitude range, based on the extracted information relating to the amplitude of the waveform.

According to the seventh or eighth aspect of the present invention, the amplitude value of the waveform is extracted as a feature of the waveform signal, and the occurrence of the body motion is determined based on the variation of the amplitude value. Therefore, the body motion can be determined through a relatively simple process, merely by monitoring a unique amplitude variation of the waveform signal.

A ninth aspect of the present invention is the biological information measurement apparatus according to the first aspect, wherein the feature extractor is configured to extract, as a feature of the waveform of the waveform signal, information relating to an amplitude of a waveform of the waveform signal in each repetition interval, and the body motion detector is configured to determine that the body motion has occurred when a difference between an amplitude value of a waveform in a first repetition interval and an amplitude value of a waveform in a second repetition interval, different from the first repetition interval, exceeds a preset second amplitude range, based on the extracted information relating to the amplitude of the waveform in each repetition interval of the waveform.

According to the ninth aspect of the present invention, the amplitude value of the waveform is extracted for each repetition interval of the waveform signal as a feature of the waveform of the waveform signal, and it is determined that the body motion has occurred when a difference in the amplitude value of the waveform between a plurality of different repetition intervals exceeds a preset range. Therefore, the occurrence of the body motion can be determined merely by monitoring the change in the amplitude value of the waveform between the repetition intervals of the waveform signal.

A tenth aspect of the present invention is the biological information measurement apparatus according to the first aspect, wherein the feature extractor is configured to extract, as a feature of the waveform of the waveform signal, information relating to a spectrum intensity of a predetermined frequency band for each preset time interval of the waveform signal, and the body motion detector is configured to determine that the body motion has occurred when the information relating to the spectrum intensity exceeds a preset range, based on the extracted information relating to the spectrum intensity.

According to the tenth aspect of the present invention, a spectrum intensity of a predetermined frequency band is detected as a feature of the waveform of the waveform signal for each fixed interval of the waveform signal, and the occurrence of the body motion is determined based on the spectrum intensity. Therefore, the occurrence of the body motion can be accurately determined by monitoring the spectrum intensity of the frequency component specific to the body motion.

An eleventh aspect of the present invention is the biological information measurement apparatus according to the first aspect, wherein the feature extractor is configured to extract, as a feature of the waveform of the waveform signal, information indicating a shape of a waveform of the waveform signal in each repetition interval, and the body motion detector is configured to determine that the body motion has occurred when a correlation value between the shape of the waveform extracted and a shape of a reference waveform stored in advance is equal to or less than a preset correlation value, based on the extracted information relating to the shape of the waveform.

According to the eleventh aspect of the present invention, the shape of the waveform of the waveform signal in each repetition interval is extracted as a feature of the waveform of the waveform signal, and the correlation value between the shape of the waveform extracted and the shape of the reference waveform is obtained for each repetition interval of the waveform signal, so that the occurrence of the body motion is determined based on the correlation value. Therefore, the occurrence of the body motion can be accurately determined by focusing on the change of the waveform shape of the waveform signal with respect to the shape of the reference waveform due to the body motion.

A twelfth aspect of the present invention is the biological information measurement apparatus according to the first aspect, wherein the feature extractor is configured to extract, as a feature of the waveform of the waveform signal, information indicating a shape of a waveform of the waveform signal in each repetition interval, and the body motion detector is configured to determine that the body motion has occurred when a correlation value between a shape of a waveform in a first repetition interval and a shape of a waveform in a second repetition interval, different from the first repetition interval, is equal to or less than a preset correlation value, based on the extracted information relating to the shape of the waveform.

According to the twelfth aspect of the present invention, the shape of the waveform of the waveform signal in each repetition interval is extracted as a feature of the waveform of the waveform signal, and the occurrence of the body motion is determined based on the correlation value between the waveform shapes in the repetition intervals. Therefore, the occurrence of the body motion can be accurately determined by focusing on the change of the waveform shape of the waveform signal between the respective repetition intervals due to the body motion.

A thirteenth aspect of the present invention is the biological information measurement apparatus according to any one of the first to eighth aspects, wherein the body motion detector is configured to periodically perform an operation of determining an occurrence of the body motion, and return to the operation of determining an occurrence of the body motion when it is not determined that the body motion has occurred continuously for a preset period of time, or when it is not determined that the body motion has occurred continuously for the preset number of cycles after the occurrence of the body motion is determined.

According to the thirteenth aspect of the present invention, when the occurrence of the body motion is detected, the body motion detector returns to the operation of determining an occurrence of a body motion only when such occurrence is not detected continuously for a predetermined period of time or continuously for a predetermined number of cycles. Therefore, the body motion detector does not instantly return to the operation of body motion detection when the occurrence of the body motion is not detected temporarily, whereby a highly stable operation of body motion detection can be performed.

A fourteenth aspect of the present invention is the biological information measurement apparatus according to any one of the first to ninth aspects, wherein the body motion detector further includes an operation control unit configured to stop power supply to at least one of the transmitter, the receiver, the feature extractor, or the body motion detector for a preset time period when the body motion occurrence is detected.

According to the fourteenth aspect of the present invention, when the body motion occurrence is detected, the power supply to each unit of the apparatus is stopped for a certain period of time, whereby it is possible to reduce power consumption waste caused by continuing the measurement under inappropriate conditions where body motion influence cannot be ignored.

A fifteenth aspect of the present invention is the biological information measurement apparatus according to the ninth aspect, wherein the body motion detector further includes an operation control unit configured to stop power supply to at least one of the transmitter, the receiver, the feature extractor, or the body motion detector from a time point when the occurrence of the body motion is detected to a time point when the body motion detector returns to the operation of determining the occurrence of the body motion.

According to the fifteenth aspect of the present invention, the power supply to each unit of the apparatus is stopped after the occurrence of the body motion is detected until the occurrence of the body motion is no longer detected. Therefore, the power supply can be stopped only during the period in which the body motion is being detected.

A sixteenth aspect of the present invention is the biological information measurement apparatus according to any one of the first to ninth aspects, further including an output unit configured to output a result of detection by the body motion detector.

According to the sixteenth aspect of the present invention, a detection result of the state of the occurrence of the body motion is output. Therefore, it is possible to, for example, reflect the detection result of the state of the occurrence of the body motion in the operation of measuring the biological information, present the detection result to the user, store the detection result in a storage, or transmit the detection result to an external apparatus; it is also possible to take various measures by using the detection result of the state of the occurrence of the body motion. For example, a result of measurement of the biological information obtained during a period in which the body motion has occurred can be discarded or unused as unreliable information. Also, it is possible to prompt the user to stop the body motion during measurement by presenting the detection result of the state of the occurrence of the body motion to the user. Furthermore, storing the state of the occurrence of the body motion in a storage, or transmitting the state of the occurrence of the body motion to an external apparatus, allows the user to understand his or her health management, or allows a healthcare worker in a remote area to monitor the user's health condition.

### Advantageous Effects of Invention

Specifically, according to each aspect of the present invention, it is possible to provide a biological information measurement apparatus, method, and program that allow for detection of a body motion of a user without the addition of another sensor device.

### BRIEF DESCRIPTION OF THE DRAWINGS

FIG. 1 is a block diagram for illustrating an application example of a biological information measurement apparatus according to an embodiment of the present disclosure.

FIG. 2 is a perspective view of an appearance of a wrist-type blood pressure monitor of an embodiment related to the biological information measurement apparatus shown in FIG. 1.

FIG. 3 is a diagram showing an example of a planar layout of first and second pulse wave sensors in a state where the blood pressure monitor shown in FIG. 2 is worn on a left wrist.

FIG. 4 is a block diagram showing an outline of a configuration of the biological information measurement apparatus according to an embodiment of the present disclosure.

FIG. 5 is a block diagram showing a detailed functional configuration of the biological information measurement apparatus shown in FIG. 4.

FIG. 6 is a diagram illustrating an example of a method of detecting a state of an occurrence of a body motion according to the embodiment of the present disclosure.

FIG. 7 is a flowchart illustrating an example of a process procedure of the biological information measurement apparatus according to the embodiment of the present disclosure using the method of detecting a state of an occurrence of a body motion illustrated in FIG. 6.

FIG. 8 is a diagram illustrating another example of the method of detecting a state of an occurrence of a body motion according to the embodiment of the present disclosure.

FIG. 9 is a diagram illustrating another example of the method of detecting a state of an occurrence of a body motion according to the embodiment of the present disclosure.

FIG. 10 is a diagram illustrating another example of the method of detecting a state of an occurrence of a body motion according to the embodiment of the present disclosure.

FIG. 11 is a diagram illustrating another example of the method of detecting a state of an occurrence of a body motion according to the embodiment of the present disclosure.

FIG. 12 is a diagram illustrating another example of the method of detecting a state of an occurrence of a body motion according to the embodiment of the present disclosure.

FIG. 13 is a block diagram showing a functional configuration of a biological information measurement apparatus according to another embodiment of the present disclosure.

FIG. 14 is a schematic diagram showing an example of a system including the blood pressure monitor shown in FIG. 2.

### DETAILED DESCRIPTION

Hereinafter, an embodiment according to one aspect of the present invention (hereinafter, also referred to as “the present embodiment”) will be described based on the drawings.

### Application Example

(Configuration)

First, an example of a scenario to which the present invention is applied will be described.

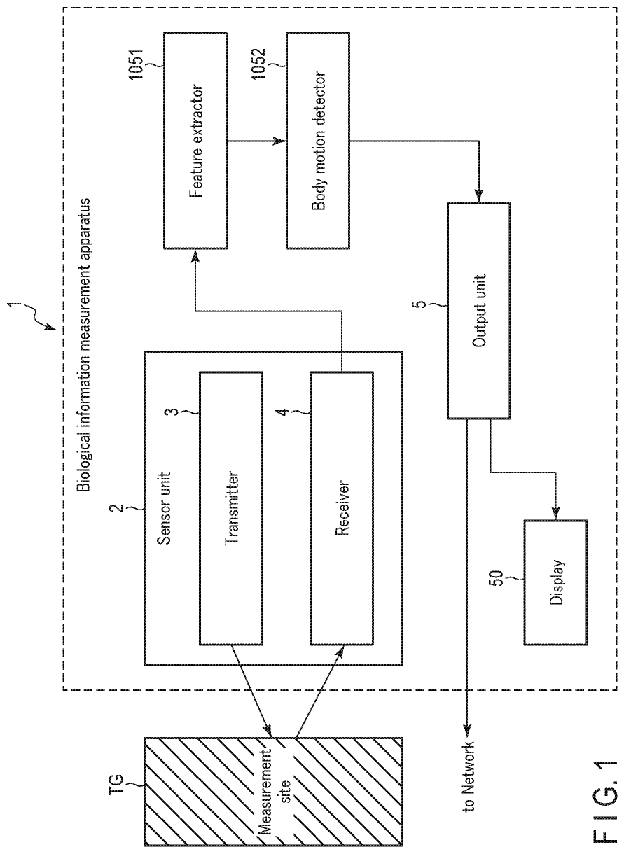

FIG. 1 schematically shows an application example of a biological information measurement apparatus according to an embodiment of the present invention.

In the example shown in FIG. 1, a biological information measurement apparatus 1 includes a sensor unit 2, a feature extractor 1051, a body motion detector 1052, an output unit 5, and a display 50. The biological information measurement apparatus 1 is disposed so that the sensor unit 2 faces a measurement site TG of a living body.

The measurement site TG is, for example, a portion of a human wrist including a radial artery. The biological information measurement apparatus 1 is, for example, a wristwatch-type wearable terminal, and is disposed so that the sensor unit 2 faces a palmar surface of the wrist when the apparatus is worn. For example, a pulse wave (or a signal related to a pulse wave) is measured as biological information. The measurement site TG may be rod-shaped such as an upper limb (wrist, upper arm, or the like) or a lower limb (ankle or the like), and may also be a trunk.

The sensor unit 2 is, for example, a pulse wave sensor that measures a pulse wave of the radial artery of the user, and includes a transmitter 3 and a receiver 4.

The transmitter 3 includes a transmission antenna element and transmitter circuitry, and transmits a radio wave as a measurement signal toward the measurement site TG.

The receiver 4 includes a reception antenna element and receiver circuitry, receives a reflected wave of the radio wave by the measurement site TG, and outputs a waveform signal of the reflected wave.

The feature extractor 1051 receives the waveform signal output from the receiver 4, generates a pulse wave signal based on the waveform signal, and then extracts a feature of a waveform from the pulse wave signal.

The body motion detector 1052 detects a state of an occurrence of a body motion based on the feature of the waveform of the pulse wave signal extracted by the feature extractor 1051. In this example, the state of an occurrence of a body motion indicates whether or not there is an occurrence of a body motion; however, it may also include a period of an occurrence of a body motion, a magnitude and direction of a body motion, and the like.

The output unit 5 outputs a detection result of the state of an occurrence of a body motion detected by the body motion detector 1052. For example, the output unit 5 generates a display message indicating that a body motion is occurring or prompting the body motion to stop based on the detection result of the state of an occurrence of a body motion, and outputs the display message to the display 50.

The display 50 includes, for example, a display and/or a speaker provided to the biological information measurement apparatus 1, and visually or auditorily presents the user with the display message output from the output unit 5. Alternatively, the display 50 may notify the user of the detection result by vibration. The display 50 may be provided separately from the biological information measurement apparatus 1 or may be omitted.

(Operation)

In the biological information measurement apparatus 1, the transmitter 3 transmits a radio wave as a measurement signal to the measurement site TG at a fixed cycle. Then, a reflected wave of the radio wave by the measurement site TG is received by the receiver 4 at the fixed cycle. The receiver 4 generates a waveform signal of the reflected wave and outputs the waveform signal to the feature extractor 1051. The radio wave to be transmitted by the transmitter 3 may be transmitted continuously or intermittently.

When the waveform signal is input from the receiver 4, the feature extractor 1051, for example, firstly converts the waveform signal into a digital signal, and then performs filtering processing for canceling an unnecessary wave component such as a noise component to generate a pulse wave signal. The pulse wave signal is a waveform signal representing the pulsation of the radial artery passing through the measurement site TG. Next, the feature extractor 1051 extracts a feature of a waveform from the pulse wave signal. For example, the feature extractor 1051 extracts an amplitude value from the waveform of the pulse wave signal. The feature of the waveform is not limited to an amplitude value, and a periodicity of the waveform, a spectrum intensity of the waveform in a predetermined frequency band, a shape of the waveform, and the like may also be extracted as a feature of the waveform. The feature extractor 1051 outputs information indicating the extracted feature of the waveform to the body motion detector 1052.

The body motion detector 1052 detects a state of an occurrence of a body motion based on the information indicating the feature of the waveform output from the feature extractor 1051. For example, the body motion detector 1052 determines an occurrence of a body motion based on whether or not the time during which the amplitude value of the waveform exceeds a threshold continues for a certain period or longer. The method of detecting a body motion is not limited to the above-described method. The occurrence of a body motion may also be detected based on, for example, whether or not the amplitude value of the waveform exceeds a range indicated by a predetermined threshold, whether or not a difference in the amplitude value between repetition intervals of the waveform exceeds a predetermined threshold, whether or not a change in the repetition cycle of the waveform exceeds a predetermined range, whether or not a spectrum intensity of a predetermined frequency band of the waveform exceeds a range indicated by a predetermined threshold, whether or not a correlation value between the shape of the detected waveform and a shape of a reference waveform or a correlation value of the waveform between respective repetition intervals exceeds a threshold.

The output unit 5 generates a display message indicating that a body motion is occurring or prompting the body motion to stop based on the information indicating the detection result of the state of an occurrence of a body motion reported by the body motion detector 1052, and outputs the display message to the display 50 for display.

The output unit 5 can also, for example, output the information indicating the detection result of the state of an occurrence of a body motion to a storage (not shown) for the purpose of storing the information in the storage, or output the information to an external apparatus via a network.

### Advantageous Effects

As described above, according to the application example, a feature of a waveform (e.g., an amplitude value) is extracted by the feature extractor 1051 from a pulse wave signal obtained by transmission and reception of a radio wave to and from the measurement site TG, and a state of an occurrence of a body motion is detected by the body motion detector 1052 based on the extracted feature of the waveform. Therefore, a body motion of a user can be detected without adding another motion sensor such as an acceleration sensor. As a result, the apparatus can be rendered simple, compact, and inexpensive.

In addition, a display message indicating that a body motion is occurring or prompting the body motion to stop, for example, is generated by the output unit 5 based on the information indicating the detection result of the body motion, and displayed on the display 50. As a result, the user can confirm his or her own motion state based on the display message and stop the body motion during measurement of the biological information.

Furthermore, the detection result of the state of an occurrence of a body motion, for example, is stored in a storage or transmitted to an external apparatus via a network by the output unit. As a result, the detection result of the state of an occurrence of a body motion can be used by a user to know the amount of movement and the like, or by a healthcare worker in a remote area to monitor the motion state of the user, for example.

It is also possible to perform processing for discarding or not using the biological information measured in a state where a body motion is detected, for example, based on the information indicating the detection result of the state of an occurrence of a body motion stored in the storage.

### First Embodiment

### Configuration Example

(1) Structure of Blood Pressure Monitor

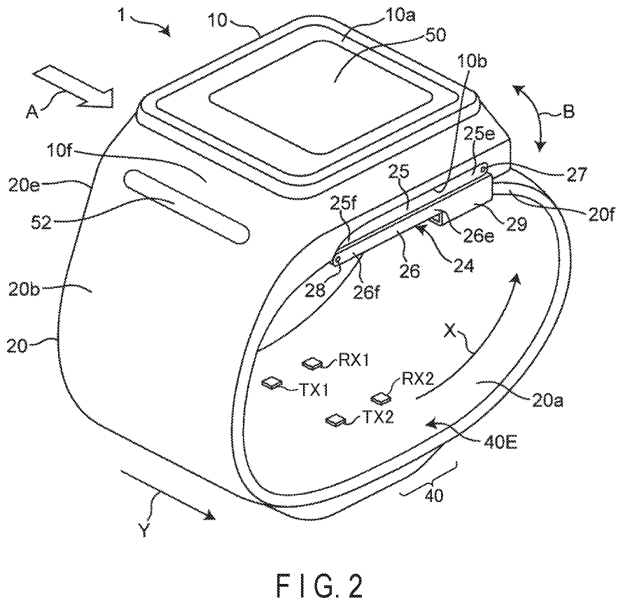

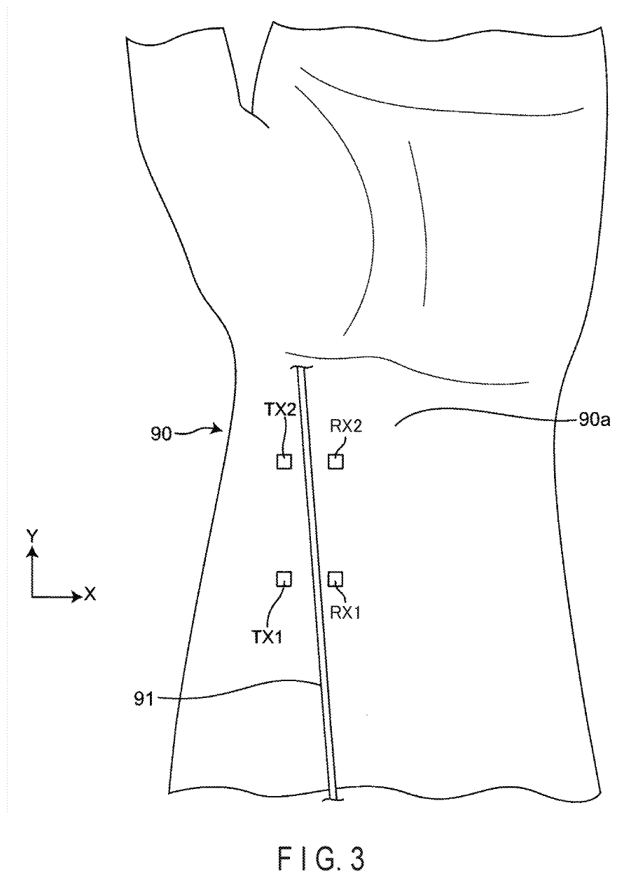

FIG. 2 is a perspective view of an appearance of a wrist-type blood pressure monitor (indicated by reference numeral 1) as the biological information measurement apparatus 1 according to a first embodiment of the present invention. FIG. 3 is a plan view schematically showing the arrangement positions of antennas TX1, RX1, TX2, and RX2 of a pulse wave sensor in a state where the blood pressure monitor 1 is worn on a left wrist 90 as a measurement site (hereinafter referred to as a “worn state”). In FIG. 3, reference numeral 90a indicates a palmar surface of the left wrist 90, and reference numeral 91 indicates the position of a radial artery 91.

As shown in FIGS. 2 and 3, the blood pressure monitor 1 broadly includes a strap 20 to be worn around the left wrist 90 of a user and a main body 10 which is integrally attached to the strap 20. As a whole, the blood pressure monitor 1 is configured to correspond to a blood pressure measurement apparatus including two pairs (two sets) of pulse wave sensors. In these figures, a pair of a transmission antenna TX1 and a reception antenna RX1 disposed on the upstream side (upper arm side) and a pair of a transmission antenna TX2 and a reception antenna RX2 disposed on the downstream side (wrist side) respectively form a pulse wave sensor.

As shown in FIG. 2, the strap 20 has an elongated belt shape so as to surround the left wrist 90 along the circumferential direction, and includes an inner peripheral surface 20a brought into contact with the left wrist 90 and an outer peripheral surface 20b opposite to the inner peripheral surface 20a. In this example, the dimension (width dimension) of the strap 20 in the width direction Y is set to about 30 mm.

In this example, the main body 10 is integrally provided at one end 20e of the strap 20 in the circumferential direction by way of integral molding. The strap 20 and the main body 10 may be separately formed, so that the main body 10 is integrally attached to the strap 20 via an engaging member (such as a hinge). In this example, an area in which the main body 10 is disposed is intended to correspond to a back surface (dorsal surface) 90b of the left wrist 90 in the worn state.

As can be seen in FIG. 2, the main body 10 has a three-dimensional shape with a thickness in a direction perpendicular to the outer peripheral surface 20b of the strap 20. The main body 10 is formed to be small and thin so as not to interfere with daily activities of the user. In this example, the main body 10 has a contour in a shape of a truncated quadrangular pyramid protruding outward from the strap 20.

A display 50 forming a display screen is provided on a top surface 10a of the main body 10 (i.e., surface farthest from the measurement site). In this example, the display 50 is formed of an organic EL (electroluminescence) display, and displays information related to blood pressure measurement, such as a result of blood pressure measurement, and other kinds of information in accordance with a control signal from a control unit (not shown). The display 50 is not limited to an organic EL display, and may be formed of another type of display such as an LCD (liquid crystal display).

A controller 52 for inputting an instruction from a user is provided on a side surface 10f of the main body 10 (a side surface on the left front side of FIG. 2). In this example, the controller 52 is formed of a push-type switch, and an operation signal corresponding to an instruction to start or stop blood pressure measurement by the user is input through the controller 52. The controller 52 is not limited to a push-type switch, and may be, for example, a pressure-sensitive (resistive) or proximity (capacitance) touch panel switch. A microphone (not shown) may also be provided to input an instruction to start blood pressure measurement with a user's voice.

A transmitter-receiver 40 constituting first and second pulse wave sensors is provided at a portion of the strap 20 between one end 20e and the other end 20f in the circumferential direction. A transmission-reception antenna group 40E including the antennas TX1, TX2, RX1, and RX2 spaced from each other in the longitudinal direction X and the width direction Y of the strap 20 is mounted on a part of the inner peripheral surface 20a corresponding to the part of the strap 20 where the transmitter-receiver 40 is arranged. In this example, the range occupied by the transmission-reception antenna group 40E in the longitudinal direction X of the strap 20 is intended to correspond to the radial artery 91 of the left wrist 90 in the worn state (see FIG. 3).

As shown in FIG. 2, a bottom surface 10b of the main body 10 (surface closest to the measurement site) and the end 20f of the strap 20 are connected to each other by a three-fold buckle 24. The buckle 24 includes a first plate-shaped member 25 disposed on the outer peripheral side and a second plate-shaped member 26 disposed on the inner peripheral side. One end 25e of the first plate-shaped member 25 is rotatably attached to the main body 10 via a connecting rod 27 extending along the width direction Y. The other end 25f of the first plate-shaped member 25 is rotatably attached to one end 26e of the second plate-shaped member 26 via a connecting rod 28 extending along the width direction Y. The other end 26f of the second plate-shaped member 26 is fixed near the end 20f of the strap 20 by a fixing unit 29. The mounting position of the fixing unit 29 in the longitudinal direction X of the strap 20 (corresponding to the circumferential direction of the left wrist 90 in the worn state) is variably set in advance in accordance with the circumferential length of the left wrist 90 of the user. Thus, the blood pressure monitor 1 (strap 20) is configured to have an approximately annular shape as a whole, and the bottom surface 10b of the main body 10 and the end 20f of the strap 20 can be opened and closed in the direction of the arrow B by the buckle 24.

When wearing the blood pressure monitor 1 on the left wrist 90, the user passes his or her left hand through the strap 20 in the direction indicated by the arrow A in FIG. 2 in a state where the buckle 24 is opened to increase the diameter of the ring of the strap 20. Then, the user adjusts the angular position of the strap 20 around the left wrist 90 to position the transmitter-receiver 40 of the strap 20 on the radial artery 91 passing through the left wrist 90. As a result, the transmission-reception antenna group 40E of the transmitter-receiver 40 comes into contact with a portion 90al of the palmar surface 90a of the left wrist 90 corresponding to the radial artery 91. In this state, the user closes and fixes the buckle 24. In this manner, the user wears the blood pressure monitor 1 (strap 20) on the left wrist 90.

In the worn state, the transmission-reception antenna group 40E of the transmitter-receiver 40 includes two transmission antennas TX1 and TX2 and two reception antennas RX1 and RX2, which are spaced from each other substantially along the longitudinal direction of the left wrist 90 (corresponding to the width direction Y of the strap 20) and the circumferential direction of the left wrist 90 (corresponding to the longitudinal direction X of the strap 20), in a manner corresponding to the radial artery 91 of the left wrist 90, as shown in FIG. 3.

In this example, the transmission antennas or the reception antennas have a pattern shape of a square of about 3 mm in length and width in a planar direction (i.e., the direction of the sheet of drawing in FIG. 3) so as to be able to emit or receive a radio wave having a frequency of 24 GHz band.

Each of the transmission antennas TX1 and TX2 has a conductive layer for emitting a radio wave (not shown). A dielectric layer is attached along a surface of the conductive layer facing the left wrist 90 (The respective transmission antennas and reception antennas have the same configuration.) In the worn state, the conductive layer faces the palmar surface 90a of the left wrist 90, and the dielectric layer serves as a spacer to keep the distance between the palmar surface 90a of the left wrist 90 and the conductive layer constant. Thus, the biological information from the left wrist 90 can be accurately measured.

The conductive layer is made of, for example, a metal (copper or the like). The dielectric layer is made of, for example, polycarbonate, whereby the relative dielectric constant of the dielectric layer is uniformly set to εr≈3.0. The relative dielectric constant refers to a relative dielectric constant at a frequency of 24 GHz band of a radio wave used for transmission and reception.

The transmission-reception antenna group 40E described above may be configured to lie flat along the planar direction. Therefore, in the blood pressure monitor 1, the strap as a whole 20 can be configured to be thin.

In FIGS. 2 and 3, the blood pressure monitor 1 including two sets of pulse wave sensors is shown. However, the number of sensors is not limited thereto. For example, three or more sets of pulse wave sensors may be dispersed along the radial artery 91, so that pulse waves are measured at three or more areas of the radial artery by these pulse wave sensors. With this configuration, the number of measurements of the pulse wave signal can be increased, allowing for improvement of the accuracy in calculating a pulse transit time (PTT), for example.

(2) Functional Configuration of Blood Pressure Monitor 1

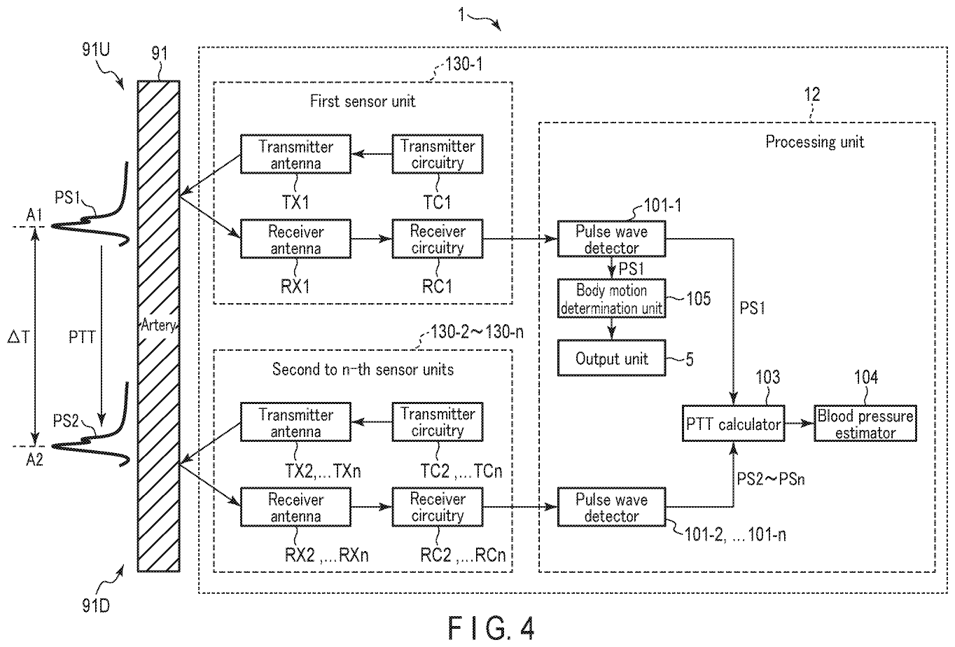

FIG. 4 is a block diagram showing a functional configuration of the blood pressure monitor 1 according to the first embodiment of the present invention.

The blood pressure monitor 1 includes a plurality of sensor units and a processing unit 12. To simplify the illustration, FIG. 4 illustrates the sensor units as a first sensor unit 130-1 and second to n-th sensor units 130-2 to 130-n. Also, FIG. 4 illustrates the artery 91 as having an upstream side (upper arm side) 91U on the upper side of the figure and a downstream side (wrist side) 91D on the lower side of the figure.

The first sensor unit 130-1 includes a pair of the transmission antenna TX1 and the reception antenna RX1, and transmitter circuitry TC1 and RC1 connected to the transmission antenna TX1 and the reception antenna RX1, respectively. The transmission antenna TX1 and the reception antenna RX1 both have directivity in the direction of the measurement site including the radial artery 91. The transmitter circuitry TC1 feeds a measurement signal to the transmission antenna TX1 at a constant cycle, thereby transmitting a radio wave of the measurement signal from the transmission antenna TX1 to the measurement site. The reception antenna RX1 receives a reflected wave of the radio wave of the measurement signal by the radial artery 91. The receiver circuitry RC1 generates a waveform signal corresponding to the reflected wave received by the reception antenna RX1 and outputs the waveform signal to the processing unit 12.

The configuration of each of the second to n-th sensor units 130-2 to 130-n is the same as that of the first sensor unit 130-1, and thus a description thereof will be omitted.

The processing unit 12 includes, for example, a hardware processor, such as a central processing unit (CPU), and a work memory, and includes pulse wave detectors 101-1, 101-2, . . . , 101-n (101-1 to 101-n), a PTT calculator 103, a blood pressure estimator 104, a body motion determination unit 105, and, as processing function units, an output unit 5 according to an embodiment. All these processing function units are implemented by causing the hardware processor to execute a program stored in a storage unit (not shown).

The pulse wave detectors 101-1 to 101-n capture the waveform signals output from the sensor units 130-1 to 130-n, respectively, to generate pulse wave signals PS1 to PSn, and output the pulse wave signals PS1 to PSn to the PTT calculator 103 and the body motion determination unit 105.

The PTT calculator 103 calculates, as a pulse transit time (PTT), a time difference between the pulse wave signals PS1 and PS2 output from any of the pulse wave detectors 101-1 to 101-n (e.g., 101-1, 101-2).

The blood pressure estimator 104 estimates a blood pressure value corresponding to the pulse transit time (PTT) calculated by the PTT calculator 103, based on the pulse transit time (PTT) calculated by the PTT calculator 103 and a correspondence equation representing a relationship between a PTT and a blood pressure value stored in a storage unit (not shown).

The body motion determination unit 105 extracts a feature of a waveform from the pulse wave signal output from the pulse wave detector 101-1. Then, based on the extracted feature of the signal waveform, the body motion determination unit 105 detects a state of an occurrence of a body motion (e.g., whether or not there is an occurrence of a body motion, a period of an occurrence of a body motion) affecting measurement of the biological information.

When it is determined that a body motion has occurred, the body motion determination unit 105 controls power supply circuitry to selectively cut power supply to the sensor units 130-1 to 130-n over a preset certain period of time from the detection time point or in a period from the detection time point until the body motion is no longer detected.

The output unit 5 generates a display message indicating that a body motion is occurring or prompting the body motion to stop, for example, based on the detection result of the state of an occurrence of a body motion by the body motion determination unit 105, so that the display message is displayed on a display (not shown).

The output unit 5 can also output information indicating the detection result of the state of an occurrence of a body motion, for example, to a storage (not shown) for the purpose of storing the information in the storage, or output the information to an external apparatus via a network. In this case, the output unit 5 may include other kinds of information, such as information indicating time, the ID of the user or the biological information measurement apparatus 1, and the acquired pulse wave signal, in the information indicating the detection result of the state of an occurrence of a body motion.

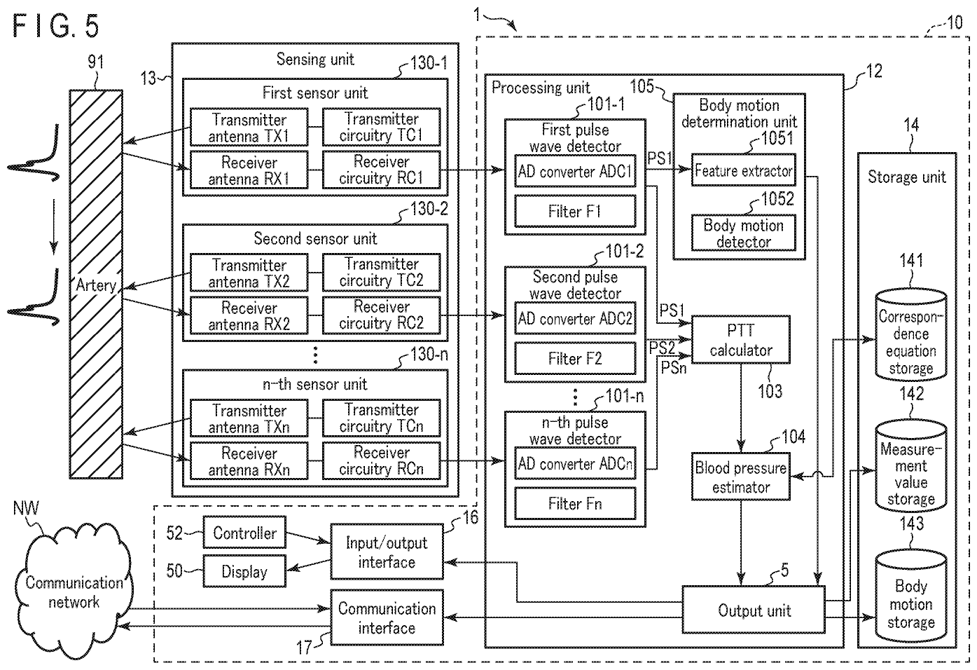

FIG. 5 is a block diagram illustrating the functional configuration of the blood pressure monitor 1 shown in FIG. 4 in more detail. In FIG. 5, the same components as those shown in FIG. 4 are denoted by the same reference numerals, and a description thereof will be omitted.

The blood pressure monitor 1 includes a sensing unit 13, a processing unit 12, a storage unit 14, an input/output interface 16, a communication interface 17, a display 50, and a controller 52. Among these components, the processing unit 12, the storage unit 14, the input/output interface 16, the communication interface 17, the display 50, and the controller 52 are provided in the main body 10.

The input/output interface 16 has, for example, a function of receiving an instruction input by a user via the controller 52 and outputting display data generated by the processing unit 12 to the display 50.

The communication interface 17 includes, for example, a wired or wireless interface, and enables transmission and reception of information to and from a terminal carried by a user, a server (not shown) on a cloud, or the like via a communication network NW. In the present embodiment, the network NW is the Internet, but is not limited thereto. The network NW may be another type of network such as an in-hospital local area network (LAN), or one-to-one communication using a USB cable or the like. The communication interface 17 may be an interface for a micro USB connector.

The storage unit 14 is a combination of a nonvolatile memory, such as an HDD (hard disk drive) or an SSD (solid state drive), that allows for write and read operations at any time, and a volatile memory, such as a RAM, as a storage medium, and includes a program storage (not shown), a correspondence equation storage 141, a measurement value storage 142, and a body motion storage 143 as storage areas necessary for implementing the present embodiment.

A correspondence equation representing a relationship between a pulse transit time (PTT) and a blood pressure value is stored in advance in the correspondence equation storage 141. The correspondence equation will be detailed later.

The measurement value storage 142 is used to store a log relating to a measurement result of a blood pressure value.

The body motion storage 143 is used to store information indicating the detection result of the state of an occurrence of a body motion.

The measurement value storage 142 and the body motion storage 143 need not necessarily be built in the biological information measurement apparatus 1, and may be provided in, for example, a mobile terminal carried by a user or an external storage device such as a server on a cloud. In this case, the blood pressure monitor 1 can access the measurement value storage 142 and the body motion storage 143 by communicating with the mobile terminal or the server via the communication network NW.

The sensing unit 13 includes a plurality of sensor units 130-1 to 130-n (hereinafter also collectively referred to as “sensor units 130”) as pulse wave sensors. As also described with reference to FIG. 4, each sensor unit 130 includes transmission antennas TX1 to TXn, transmitter circuitry TC1 to TCn that transmits a radio wave through the transmission antennas, reception antennas RX1 to RXn, and receiver circuitry RC1 to RCn that receives a reflected wave through the reception antennas.

As also described with reference to FIG. 4, the processing unit 12 includes a hardware processor, such as a CPU, and a work memory, and includes a plurality of pulse wave detectors 101-1 to 101-n corresponding to the sensor units 130-1 to 130-n, a PTT calculator 103, a blood pressure estimator 104, a body motion determination unit 105, and an output unit 5.

The pulse wave detectors 101-1 to 101-n include AD converters ADC1 to ADCn and filters F1 to Fn, respectively. The AD converters ADC1 to ADCn convert the waveform signals output from the receiver circuitry RC1 to RCn, respectively, into digital signals. The filters F1 to Fn perform filtering processing for canceling noise components, for example, on the waveform signals converted into digital signals, thereby outputting pulse wave signals PS1 to PSn. The pulse wave signals represent pulsation of the radial artery 91 passing through the left wrist 90 at the arrangement position of the above transmission-reception antennas.

The body motion determination unit 105 includes a feature extractor 1051 and a body motion detector 1052.

The feature extractor 1051 receives the pulse wave signal PS1 output from at least one of the pulse wave detectors 101-1 to 101-n (in this example, the pulse wave detector 101-1), and extracts a feature of a waveform from the pulse wave signal PS1. The process of extracting the feature of the waveform will be detailed later.

The body motion detector 1052 receives information indicating the feature of the waveform extracted by the feature extractor 1051, and detects a state of an occurrence of a body motion affecting measurement of the pulse wave. The process of detecting the state of an occurrence of a body motion will also be detailed later.

### Operation Example

(1) Measurement of Pulse Wave and Estimation of Blood Pressure

Next, an operation example of the blood pressure monitor 1 according to an embodiment of the present invention will be described.

The blood pressure monitor 1 transmits, using the first sensor units 130-1 to 130-n, radio waves as measurement signals at a constant cycle from the transmitter circuitry TC1 to TCn toward a plurality of different areas of the measurement site including the radial artery 91 via the transmission antennas TX1 to TXn. Then, reflected waves of the respective radio waves by the measurement site are received through the reception antennas RX1 to RXn, and waveform signals corresponding to the reflected waves are generated by the receiver circuitry RC1 to RCn. These waveform signals are input to the pulse wave detectors 101-1 to 101-n of the processing unit 12.

The pulse wave detectors 101-1 to 101-n of the processing unit 12 perform processing for converting into digital signals and filtering processing for canceling noise components on the waveform signals output from the receiver circuitry RC1 to RCn, thereby obtaining pulse wave signals PS1 to PSn. The pulse wave signals PS1 to PSn are input to the PTT calculator 103.

The PTT calculator 103 calculates a time difference between any pulse wave signals (e.g., PS1 and PS2) among the input pulse wave signals PS1 to PSn as a pulse transit time (PTT). For example, in the example shown in FIG. 4, a time difference Δt between a peak A1 of the amplitude of the pulse wave signal PS1 and a peak A2 of the amplitude of the pulse wave signal PS2 is calculated as a pulse transit time (PTT). The calculation result of the pulse transit time (PTT) is input to the blood pressure estimator 104.

The blood pressure estimator 104 performs processing for estimating a blood pressure value corresponding to the pulse transit time (PTT), calculated by the PTT calculator 103 based on the pulse transit time (PTT) calculated by the PTT calculator 103, and a correspondence equation representing a relationship between a PTT and a blood pressure value stored in the correspondence equation storage 141 of the storage unit 14.

For example, when a pulse transit time is represented as DT and blood pressure is represented as EBP, a correspondence equation Eq is provided as a known fractional function including a term of 1/DT², as shown by:

EBP=α/DT²+β (Eq. 1)

(where α and β each represent a known coefficient or constant).

As the correspondence equation Eq, another known correspondence equation, such as an equation including a term of 1/DT and a term of DT in addition to the term of 1/DT², may be employed, as shown by:

EBP=α/DT²+β/DT+γDT+δ (Eq. 2)

(where α, β, γ, and δ each represent a known coefficient or constant).

The estimate value of the blood pressure calculated by the blood pressure estimator 104 is stored as a blood pressure log in the measurement value storage 142 via the output unit 5, for example. The estimate value of the blood pressure may be displayed on the display 50 by the output unit 5 via the input/output interface 16, for example. However, the estimate value of the blood pressure can be a mere reference value to be used as a trigger for prompting a more accurate blood pressure measurement.

For example, if the blood pressure monitor 1 has a further blood pressure measurement function adopting an oscillometric method, in addition to the blood pressure estimation function based on the PTT, it may determine whether or not the estimate value of the blood pressure based on the PTT exceeds a range indicated by a threshold, and, when determining that the estimate value of the blood pressure exceeds said range, activate the blood pressure measurement function adopting the oscillometric method to measure blood pressure more accurately. If the blood pressure monitor 1 does not have the blood pressure measurement function adopting the oscillometric method, a message indicating that the estimate value of the blood pressure based on the PTT exceeds the range indicated by a threshold may be displayed on the display 50 to prompt the user to perform blood pressure measurement using a separately prepared oscillometric blood pressure monitor.

(2) Detection and Output of State of Occurrence of Body Motion

In the blood pressure monitor 1, a process of detecting and outputting a state of an occurrence of a body motion is performed in parallel with the above-described process of calculating the PTT and estimating the blood pressure, as will be described below.

That is, the body motion determination unit 105 of the blood pressure monitor 1 acquires the pulse wave signal PS1 from any one of the pulse wave detectors 101-1 to 101-n (e.g., the pulse wave detector 101-1). Then, the body motion determination unit 105 extracts a feature of a waveform from the pulse wave signal PS1, and detects a state of an occurrence of a body motion affecting measurement of the biological information based on the extracted feature of the signal waveform. Multiple kinds of methods can be considered as a method of detecting a state of an occurrence of a body motion by extracting a feature of a waveform from the pulse wave signal. These methods will be detailed later.

When the occurrence of a body motion is detected, information indicating the detection result is passed from the body motion determination unit 105 to the output unit 5. Based on the detection result, the output unit 5 generates a display message indicating that a body motion is occurring or prompting the body motion to stop, for example, and sends the display message to the display 50 via the input/output interface 16. Thus, the display message is displayed on the display 50. As a result, the user can confirm his or her own motion state based on the display message and stop the body motion during the period of measurement of the pulse wave.

At the same time as or instead of displaying the display message on the display 50, a voice message such as “Measurement cannot be made. Don't move.” or a warning sound may be output from a speaker provided in the display 50. Instead of the warning sound, the blinking of a light or a vibration may also be used.

For example, the output unit 5 stores the detection result of the state of the occurrence of the body motion in the body motion storage 143. Therefore, by reading the information of the stored detection result and displaying it on the display 50 according to the user's operation, for example, the user can use the information to know whether or not there is a movement, the amount of movement, and the like. Evaluation of the degree of a body motion during sleep based on the detection result of the state of the occurrence of the body motion at night can also be used to evaluate the quality of sleep.

Furthermore, the information indicating the detection result of the state of the occurrence of the body motion is, for example, transmitted by the output unit 5 to an external apparatus via a network. In this case, the ID of the user or the blood pressure monitor 1, the measurement time, the waveform of the measured pulse wave, the calculated estimate value of the blood pressure, and the like are either included in or added to the information indicating the detection result of the state of the occurrence of the body motion, and subsequently transmitted. As a result, a family member or a healthcare worker in a remote area can monitor the motion state of the user. This is effective when remotely monitoring an elderly person, for example.

(3) Detection of State of Occurrence of Body Motion

(3-1) First Detection Method

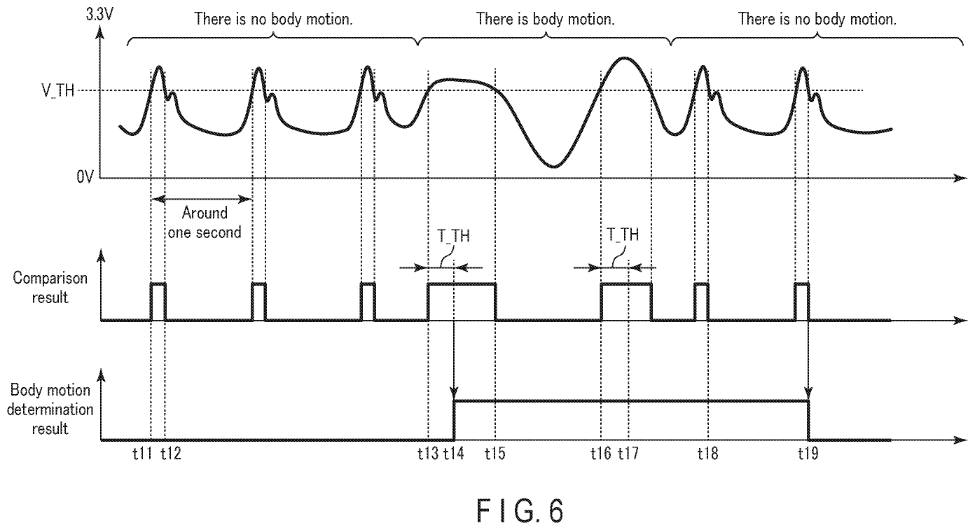

FIG. 6 is a waveform diagram for illustrating a first method of detecting a body motion.

In the first detection method, an amplitude value of a waveform is extracted as a feature of a waveform of a received pulse wave signal, and an occurrence and termination of a body motion affecting measurement of a pulse wave are detected based on the extracted amplitude value.

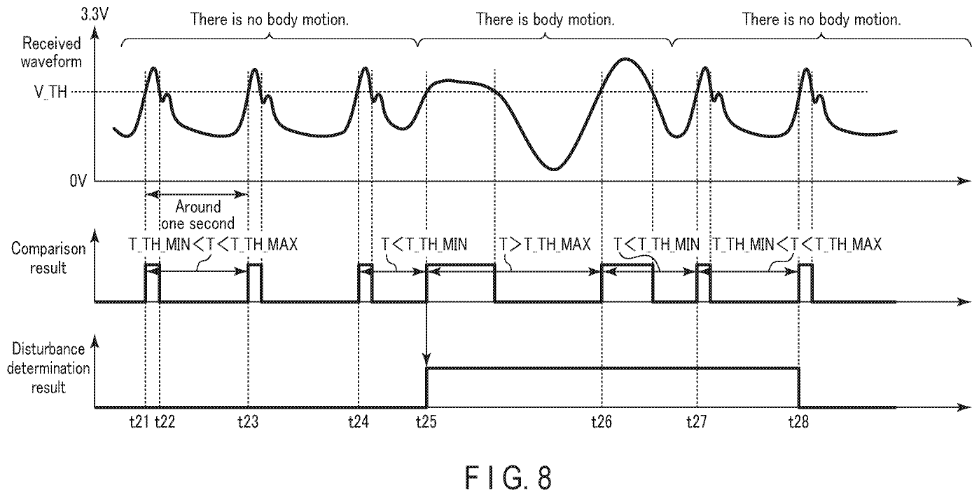

As shown in FIG. 6, a pulse wave signal is detected as a change in a voltage value with respect to a time axis. In general, it is known that a cycle is about one second when a pulse wave of the radial artery 91 is measured. The signal shown in FIG. 6 is merely an example for convenience in illustrating the detection method according to the embodiment, and the present invention is not limited thereto. The same applies to FIGS. 8 to 12 used to illustrate second to sixth detection methods.

In the first detection method, it is determined that a body motion has occurred when the time during which the amplitude value of the received pulse wave signal exceeds a preset threshold V_TH is longer than a preset time threshold T_TH. That is, when the time during which the reception signal intensity (voltage or the like) of a reflected wave exceeds a preset intensity threshold V_TH runs beyond the time threshold T_TH, it is determined that a body motion has occurred.

On the other hand, when the time during which the amplitude value of the pulse wave signal exceeds the threshold V_TH becomes shorter than the time threshold T_TH, it is determined that the body motion has stopped.

The body motion determination unit 105 of the processing unit 12, for example, turns on a body motion determination flag during a period in which it is determined that a body motion is occurring, and turns off the body motion determination flag during a period in which an occurrence of a body motion is not detected, thereby indicating the state of the occurrence of the body motion.

The above operation will be described in more detail. First, the blood pressure monitor 1 is in a state of performing a body motion occurrence detection operation (a state of monitoring whether or not an occurrence of a body motion is newly detected). In FIG. 6, the signal intensity exceeds the intensity threshold V_TH at a time point t11. However, at a time point t12 before passing the time threshold T_TH, the signal intensity of the pulse wave signal decreases to less than the intensity threshold V_TH. Therefore, it is determined that no body motion affecting the pulse wave measured at this time has occurred.

Thereafter, it is determined that the time during which the signal intensity of the pulse wave signal exceeds the intensity threshold V_TH at a time point t13, and the signal intensity of the pulse wave signal exceeds the intensity threshold V_TH at a time point t14, has exceeded the time threshold T_TH (threshold-exceeded time>T_TH). It is presumed that this is because a noise component caused by the body motion is superimposed on the pulse wave signal (a low-frequency component of the body motion is superimposed on the waveform=there is a body motion). Therefore, it is determined that a body motion affecting measurement of the pulse wave has occurred, and the body motion determination flag is turned on at the time point t14. Since the body motion determination flag is on, the blood pressure monitor 1 shifts to a state of monitoring non-detection of a body motion occurrence rather than monitoring detection of such.

Subsequently, at a time point t15, the signal intensity of the pulse wave signal falls below the intensity threshold V_TH. However, since the condition for determining non-detection is not satisfied, the body motion determination flag is maintained to be on. At a time point t16, the signal intensity of the pulse wave signal exceeds the intensity threshold V_TH again, and at a time point t17, the intensity threshold-exceeded time exceeds the time threshold T_TH again (threshold-exceeded time>T_TH). It is determined that the body motion is continuing during this time, and the body motion determination flag is maintained to be on. Thereafter, at a time point t18, the time during which the signal intensity of the pulse wave signal exceeds the intensity threshold V_TH becomes less than the time threshold T_TH (threshold-exceeded time<T_TH). This is determined to be a phenomenon (i.e., no body motion) due to disappearance (or reduction) of the noise component caused by the body motion that is superimposed on the pulse wave signal.

In regard to the first detection method, however, it is not determined that the body motion has stopped immediately at the time point t18 in FIG. 6; rather, at a time point t19, that is, after it is determined that the intensity threshold-exceeded time becomes less than T_TH (threshold-exceeded time<T_TH) two consecutive times, the body motion determination flag is turned off. When the body motion determination flag is turned off, the blood pressure monitor 1 returns to the operation of detecting an occurrence of a body motion again. That is, in the example shown in FIG. 6, the body motion determination flag is turned on when a result of comparison between the signal intensity of the pulse wave signal and the intensity threshold is “High” for a certain period of time or longer, and the body motion determination flag is turned off after a certain period of time in the case of the stopping method.

As described above, in the first detection method, it is not immediately determined that the body motion has stopped when the time during which the amplitude of the waveform of the pulse wave signal exceeds the threshold V_TH becomes less than the time threshold T_TH, but after it is confirmed that the same situation is detected stably for a certain time (when “threshold-exceeded time<constant value” continues N_TH times (twice, in FIG. 6) or more). Therefore, it is possible to reduce unnecessary processing such as the switching of display or power supply due to the frequent switching of the body motion determination flag between ON and OFF. In the first detection method, the number of times that the time during which the amplitude value of the pulse wave signal continuously exceeds the threshold V_TH becomes less than the time threshold T_TH, can be set discretionarily; thus it can be increased to, for example, three or four times, or set to a single time.

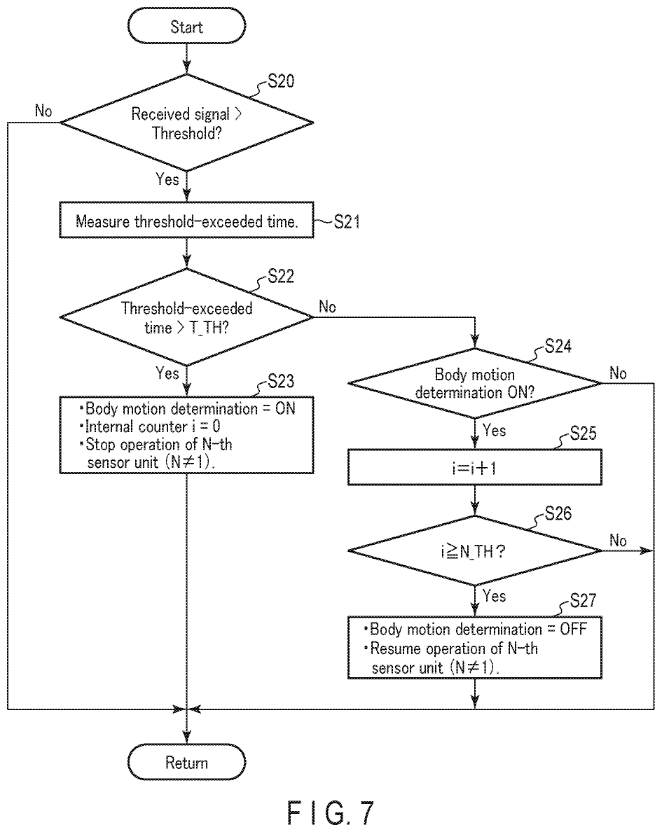

FIG. 7 is a flowchart illustrating an example of a process procedure and process content of the blood pressure monitor 1 adopting the first detection method.

Under the control of the body motion detector 1052, the processing unit 12 of the blood pressure monitor 1 firstly determines in step S20 whether or not an amplitude value of a waveform of a pulse wave signal exceeds the preset threshold V_TH. If the amplitude value does not exceed the preset threshold V_TH, the process ends.

If it is determined in step S20 that the amplitude value of the pulse wave signal exceeds the threshold V_TH, the processing unit 12 measures the time during which the amplitude value of the pulse wave signal exceeds the threshold V_TH in step S21 under the control of the body motion detector 105.

In step S22, determination is made as to whether or not the time during which the amplitude value of the pulse wave signal exceeds the threshold V_TH exceeds the time threshold T_TH. If it is determined that the time threshold T_TH is exceeded, the body motion detector 1052 proceeds to step S23.

Next, in step S23, the processing unit 12 turns on the body motion determination flag, sets an internal counter i to 0, and stops the operation of all the sensor units 130-2 to 130-n except the first sensor unit 130-1 that performs determination of a body motion, under the control of the body motion determination unit 105. The processing function of stopping the operation of the sensor units 130-2 to 130-n will be detailed in a second embodiment described later.

On the other hand, if it is determined in step S22 that the time during which the amplitude value of the pulse wave signal exceeds the threshold V_TH does not exceed the time threshold T_TH, the processing unit 12 proceeds to step S24.

In step S24, the processing unit 12 determines whether or not the current body motion determination flag is ON. If the current body motion determination is OFF, the process ends. If the current body motion determination is ON, the processing unit 12 counts up the internal counter i in step S25, and proceeds to step S26. In step S26, the processing unit 12 determines whether or not the value of the internal counter i is larger than a number-of-times threshold N_TH. If the value of the internal counter i is smaller than the number-of-times threshold N_TH, the process ends. If the value of the internal counter i is equal to or larger than the number-of-times threshold N_TH, the process proceeds to step S27.

In step S27, the processing unit 12 turns off the body motion determination flag and resumes the power supply to the sensor units whose operation has been stopped, to resume the operation of the sensor units, under the control of the body motion determination unit 105. The blood pressure monitor 1 returns to the state of performing the operation of detecting an occurrence of a body motion again.

In this manner, a state of an occurrence of a body motion can be detected by a relatively simple method, which is to evaluate an amplitude value of a waveform of a pulse wave signal, without providing an additional sensor device such as an acceleration sensor.

A value fixedly set in advance as an initial value may be used as each threshold for the detection of a body motion, or the threshold may be automatically calculated from an average value obtained when a pulse wave is properly acquired. For example, when a calibration mode is executed, data when there is no change in the waveform for a certain period of time, or data having a high correlation between a PTT value and blood pressure may be automatically extracted.

For the first detection method, a method that focuses on the time during which the amplitude value of the waveform of the pulse wave signal exceeds the threshold V_TH is described above; however, the first detection method may focus on the time during which the amplitude value of the waveform of the pulse wave signal is equal to or less than the threshold V_TH in FIGS. 6 and 7. That is, when the time during which the amplitude value is continuously equal to or less than V_TH is shorter than a second time threshold T′_TH set in advance, it can be determined that a body motion has occurred. The second time threshold T′_TH may be set separately from the time threshold T_TH, or may be obtained as a value by subtracting the time threshold T_TH from the cycle (about one second). The determination conditions may be reversed by reversing the polarity of the signal. That is, when the polarity is reversed, the time during which the amplitude value exceeds the threshold V_TH, which is focused on in FIGS. 6 and 7, can be rephrased as the time during which the amplitude value becomes equal to or less than the threshold V_TH. At this time, it can be determined that a body motion has occurred when the time during which the amplitude value is continuously equal to or less than the threshold V_TH is longer than the preset time threshold T_TH. Likewise, when the polarity is reversed, it can also be determined that a body motion has occurred when the time during which the amplitude value continuously exceeds the threshold V_TH is shorter than the preset second time threshold T′_TH.

(3-2) Second Detection Method

FIG. 8 is a waveform diagram for illustrating a second method of detecting a body motion. In the second detection method, a state of an occurrence of a body motion affecting measurement of a pulse wave is detected based on a repetition cycle of a received pulse wave signal.

For example, in the second detection method, when a repetition cycle of a received pulse wave signal exceeds a preset time range, that is, when a waveform interval is outside a predetermined range, it is determined that a body motion has occurred. When the waveform interval falls within a preset range, it is determined that the body motion has stopped. For example, a time point at which the amplitude value of the pulse wave exceeds the preset threshold V_TH can be set as a reference point in determining the repetition cycle of the waveform.

In FIG. 8, the signal intensity exceeds the intensity threshold V_TH at a time point t21. Thereafter, the signal intensity falls below V_TH at a time point t22. Next, at t23, the signal intensity exceeds V_TH again. In this example, a time interval between the time point t21 corresponding to the rising of a peak of the waveform and the time point t23 corresponding to the next rising of a peak of the waveform is set as a repetition cycle of the waveform. At the time point t23, the repetition cycle is within a preset range, that is, a range of larger than a minimum threshold T_TH_MIN and smaller than a maximum threshold T_TH_MAX (T_TH_MIN<T<T_TH_MAX), and it is therefore determined that no body motion is affecting measurement of a pulse wave (i.e., it is determined that the body motion is small enough to be acceptable).

When the repetition cycle of the waveform is continuously observed, the interval between t24 and t25 becomes smaller than the minimum threshold T_TH_MIN (T<T_TH_MIN). Therefore, at t25, it is determined that noise caused by a body motion is superimposed (a low-frequency component of the body motion is superimposed on the waveform=there is a body motion), and the body motion determination flag is turned on. Subsequently, since the interval between t25 and t26 is larger than the maximum threshold T_TH_MAX (T>T_TH_MAX), it is determined that noise is still superimposed at t26, and the body motion determination flag is kept ON. Since the interval between t26 and t27 is smaller than T_TH_MIN (T<T_TH_MIN), the body motion determination flag remains ON. Next, since the interval between t27 and t28 falls within the preset range (T_TH_MIN<T<T_TH_MAX) at t28, it is determined that the body motion has disappeared to an acceptable level (i.e., it is determined that there is no body motion), and the body motion determination flag is reset to OFF.

Regarding the determination of the stoppage of the body motion by the second detection method, the example in FIG. 8 illustrates that the body motion determination flag is turned off immediately after the waveform interval is determined to be within the predetermined range. However, the body motion determination flag may be reset from ON to OFF when the waveform interval is within the predetermined range continuously multiple times, as in the first detection method.

(3-3) Third Detection Method

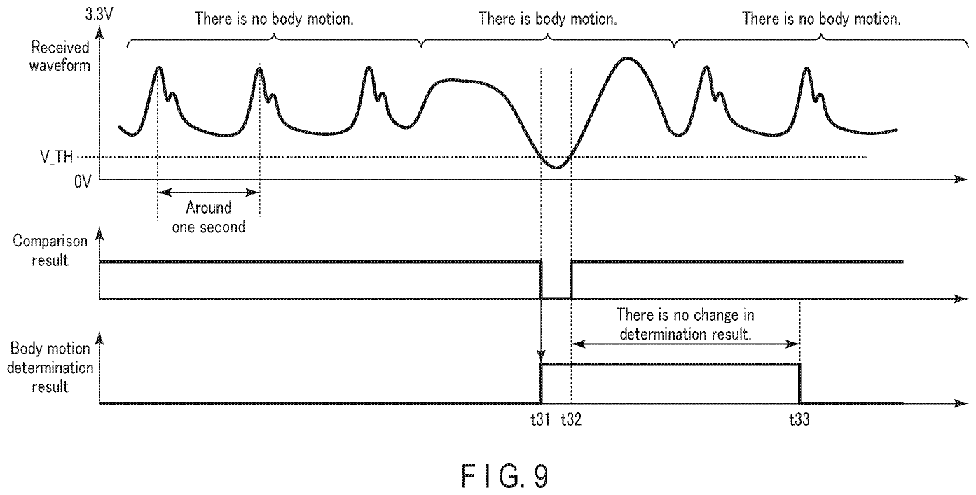

FIG. 9 is a waveform diagram for illustrating a third method for detecting a body motion.

In the third detection method, a state of an occurrence of a body motion affecting measurement of a pulse wave is detected based only on an amplitude value of a received pulse wave signal.

In the third detection method, when an amplitude value of a received pulse wave signal exceeds a preset amplitude-value range, it is determined that a body motion has occurred. Also, in the third detection method, when the range of the amplitude value is continuously within a predetermined range for a certain period of time, it is determined that the body motion has stopped.

In regard to the third detection method, the signal intensity has a value lower than the intensity threshold V_TH at the time point t31, as shown in FIG. 9. Thus, it is presumed that a noise component caused by the body motion is superimposed on the pulse wave signal (a low-frequency component of the body motion is superimposed on the waveform=there is a body motion), it is determined that a body motion affecting measurement of a pulse wave has occurred, and the body motion determination flag is turned on.

The signal intensity of the pulse wave signal exceeds the intensity threshold V_TH at the time point t32, falling within the acceptable range. However, in this example, the body motion determination flag is not reset to OFF immediately, but at the time point t33 after it is determined that the signal intensity of the pulse wave signal is continuously within an acceptable range for a certain period of time (i.e., determined that there is no change in the determination result for a certain period of time).

In the method described above, it is determined that a body motion has occurred when the amplitude value of the waveform of the pulse wave signal exceeds the preset amplitude-value range. However, it may be determined that a body motion has occurred when the amplitude value of the waveform of the pulse wave signal does not exceed the preset second amplitude-value range. That is, when it is determined that the acquired pulse wave signal does not have a sufficient amplitude, it can be presumed that a noise component caused by a body motion is superimposed. As described above, if the polarity of the signal is reversed, the detailed determination conditions may be reversed.

(3-4) Fourth Detection Method

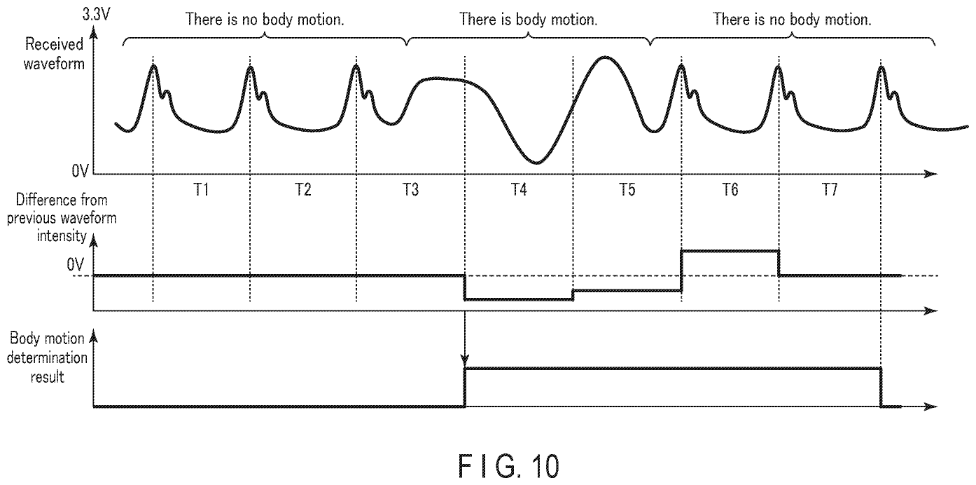

FIG. 10 is a waveform diagram for illustrating a fourth detection method for detecting a body motion.

In the fourth detection method, a state of an occurrence of a body motion affecting measurement of a pulse wave is detected based on a difference between amplitude values of waveforms in respective repetition intervals.

In the fourth detection method, when a difference between an amplitude value of a waveform in the first repetition interval and an amplitude value of a waveform in the second repetition interval exceeds a preset range, it is determined that a body motion has occurred. Also, in the fourth detection method, it is determined that the body motion has stopped when the difference in the amplitude value between the repetition intervals is within a preset range. For example, the repetition interval may be set based on the rising of the peak of the wave, as shown in FIG. 8 regarding the second detection method, or based on a generally known cycle of a pulse wave.

In regard to the fourth detection method, a difference in the peak value between an interval T2 and an interval T1, for example, which is one interval ahead in time, is evaluated, in the interval T2, as the difference in the amplitude value, as shown in FIG. 10. The difference in the peak value between the interval T1 and the interval T2 is within an acceptable range, and it is determined that there is no occurrence of a body motion. The same applies to a difference in the peak value between the interval T2 and an interval T3. However, the signal intensity of the pulse wave signal decreases greatly in an interval T4, which makes the difference in the peak value between the interval T4 and the previous interval T3 too large to be ignored. Accordingly, it is determined that the influence of the body motion on the pulse wave is large, and the body motion determination flag is turned on. In an interval T5 and an interval T6, the body motion determination flag is kept on based on the difference in the peak value from the previous interval. Since there is no difference between the peak values in an interval T7, the body motion determination flag is reset to OFF at the end of the interval T7.

(3-5) Fifth Detection Method

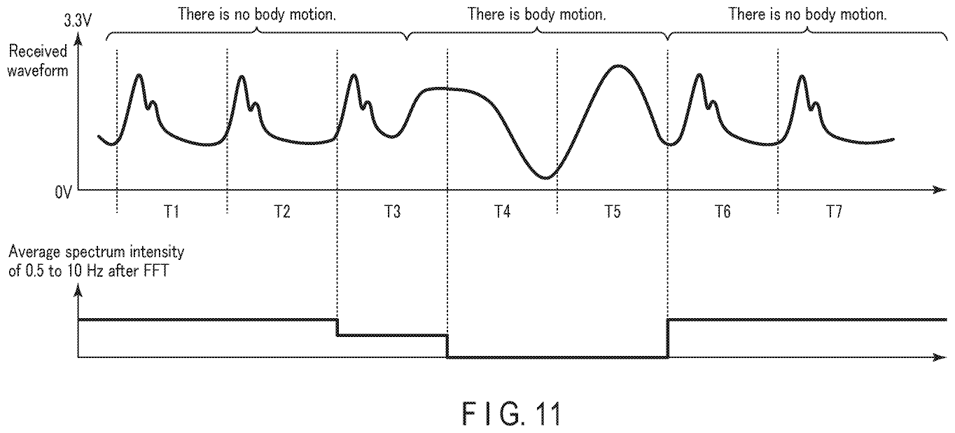

FIG. 11 is a waveform diagram for illustrating a fifth detection method for detecting a body motion.

In the fifth detection method, a state of an occurrence of a body motion affecting measurement of a pulse wave is detected based on a spectrum intensity of a predetermined frequency band of a received pulse wave signal in each preset time interval.

In the fifth detection method, spectrum analysis such as fast Fourier transform (FFT) is performed on a received waveform cut out at one-second intervals, for example, and a frequency spectrum intensity of a band including the frequency of a pulse wave (a pulse wave is usually 0.5 to 10 Hz) is calculated. When the spectrum intensity of the frequency band or an average value of the intensity exceeds a predetermined range, it is determined that a body motion has occurred. Also, in the fifth detection method, it is determined that the body motion has stopped when the spectrum intensity or the average value of the intensity is continuously within a predetermined range N times.

In regard to the fifth detection method, the spectrum intensity in the frequency band of 0.5 to 10 Hz decreases in the interval T3, and the spectrum intensity is a very small value in the intervals T4 to T5, as shown in FIG. 11. It is presumed that this is because a low-frequency component caused by the body motion is superimposed on the pulse wave and the influence of the body motion has reached a non-negligible level. In one example, the body motion determination flag is set to ON in the intervals T4 to T5.

(3-6) Sixth Detection Method

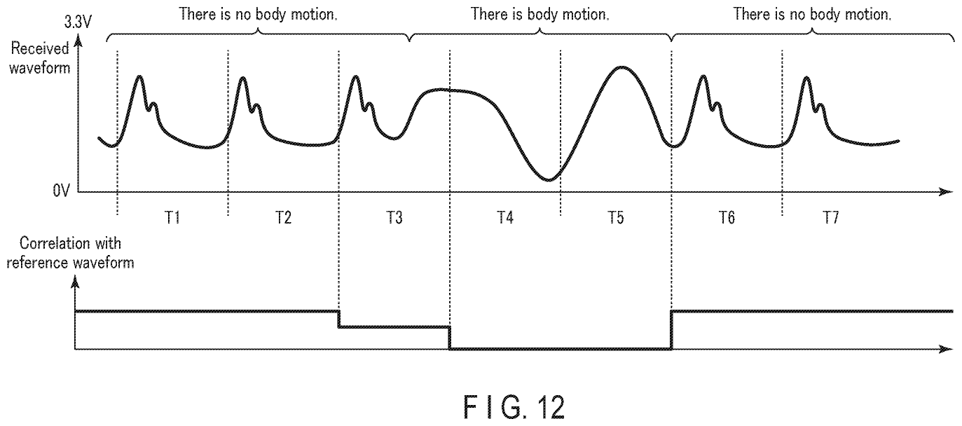

FIG. 12 is a waveform diagram for illustrating a sixth detection method for detecting a body motion.

In the sixth detection method, a state of an occurrence of a body motion affecting measurement of a pulse wave is detected based on a shape of a waveform of a received pulse wave signal in each repetition interval.

In the sixth detection method, a correlation value between a shape of a waveform of a pulse wave signal in a certain repetition interval and a reference waveform stored in advance is obtained, and when the correlation value is equal to or less than a preset correlation value, it is determined that a body motion has occurred. As another detection method, a correlation value between a shape of a waveform of a pulse wave signal in a certain repetition interval and a shape of a waveform of a pulse wave signal in another repetition interval (e.g., an interval in which it is known that no body motion has occurred), that is, autocorrelation is obtained, and when the correlation value is equal to or less than a preset correlation value, it is determined that a body motion has occurred.

In the sixth detection method, when a correlation value between a shape of a waveform in any interval and a shape of a reference waveform or an autocorrelation value of a shape of a waveform between two different intervals exceeds a preset correlation value, it is determined that the body motion has stopped. For example, the repetition interval may be set based on the rising of the peak of the waveform, as shown in FIG. 8 regarding the second detection method, or based on a generally known cycle of a pulse wave. Since the method of obtaining the correlation value is generally known, it will not be detailed here.

In regard to the sixth detection method, the correlation value becomes small in the interval T3, and the correlation value becomes very small in the intervals T4 to T5, as shown in FIG. 12, for example. It is presumed that this is because a noise component (low-frequency component) caused by the body motion is superimposed on the pulse wave and the influence thereof has reached a non-negligible level. In one example, the body motion determination flag is set to ON in the intervals T4 to T5.

It is not always necessary to provide all the first to sixth detection methods described above, and any one of the methods may be provided. Each of the first to sixth detection methods may be used by discretionarily selecting and combining a method of detecting a body motion and a method of detecting a stop of the body motion.

### Operational Advantage of First Embodiment

In the first embodiment, the feature extractor 1051 extracts a feature of a waveform from the pulse wave signal PS1 output from the pulse wave detector 101-1, and the body motion detector 1052 detects a state of an occurrence of a body motion affecting measurement of a pulse wave based on the extracted feature of the waveform, as detailed above. Therefore, a body motion of a user can be detected by using an existing sensor without adding another motion sensor such as an acceleration sensor. As a result, the apparatus can be rendered simple, compact, and inexpensive.

The output unit 5 generates a display message, for example, indicating that a body motion is occurring or prompting the body motion to stop, based on the information indicating the detection result of the body motion, so that the display message is displayed on the display 50. As a result, the user can confirm his or her own motion state based on the display message and stop the body motion during measurement of the biological information.

Also, the output unit stores, for example, log information indicating a detection result of a state of an occurrence of a body motion in the body motion storage 143 in the storage unit 14, and transmits the log information to an external apparatus via a network. Therefore, for example, the user can make use of the detection result of the state of the occurrence of the body motion to know the amount of movement or the like, or a family member or a healthcare worker in a remote area can monitor the state of the motion of the user.

It is also possible to perform processing such as discarding or not using a blood pressure value measured in a state where a body motion is detected, for example, based on the log information indicating the detection result of the state of the occurrence of the body motion stored in the body motion storage 143.

### Second Embodiment

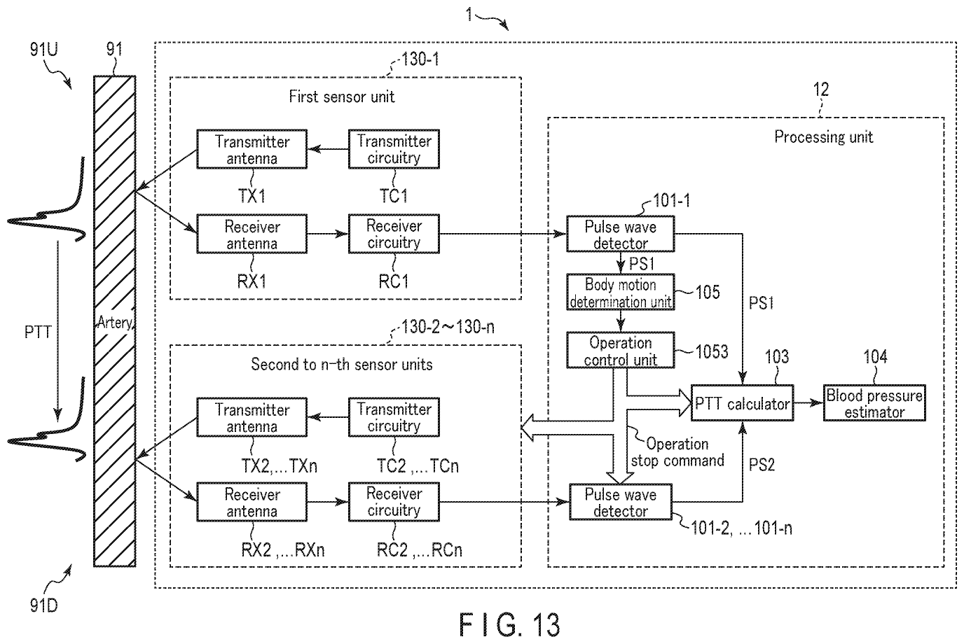

FIG. 13 is a block diagram showing a functional configuration of a blood pressure monitor 1 according to a second embodiment of the present invention. In FIG. 13, the same components as those shown in FIG. 4 are denoted by the same reference numerals, and a detailed description thereof will be omitted.

The processing unit 12 is provided with an operation control unit 1053. The operation control unit 1053 detects a period in which a body motion is detected based on the detection result of the occurrence of the body motion by the body motion determination unit 105. Then, in the detection period, power supply circuitry (not shown) is controlled so as to cut power supply to each of the sensor units 130-2 to 130-n except the first sensor unit 130-1. Also, the operation control unit 1053 stops the processing operations of the pulse wave detectors 101-2 to 101-n corresponding to the sensor units 130-2 to 130-n to which power supply is cut off and the processing operation of the PTT calculator 103.

With the above configuration, power consumption by each of the sensor units 130-2 to 130-n except the first sensor unit 130-1 and power consumption by the processing operations of the pulse wave detectors 101-2 to 101-n and the PTT calculator 103 can be set to zero during the period in which an occurrence of a body motion is detected, thereby rendering it possible to suppress the battery consumption and extend the battery life.

The operation control unit 1053 is not limited to the above-described processing operation. The operation control unit 1053 may, for example, set an operation stop period having a preset length from the detection time point, and during the operation stop period, cut the power supply to all the sensor units 130-1 to 130-n in the sensing unit 13, and also stop the operations of all the pulse wave detectors 101-1 to 101-n and the PTT calculator 103 in the processing unit 12 when an occurrence of a body motion is detected. Thereby, the battery energy can be saved more effectively. Hereinafter, the operation modes in which power supply is controlled by the operation control unit 1053 are also collectively referred to as a “power-saving mode.”

A log relating to the operation stop period may be stored in a log storage in the storage unit 14. This renders it possible to calculate the total body motion time during the measurement period.

### Operational Advantage of Second Embodiment

In the second embodiment, the operation control unit 1053 controls an operation of a predetermined functional unit of the blood pressure monitor 1 based on the state of the occurrence of the body motion detected by the body motion determination unit 105, as detailed above. For example, when it is determined that a body motion affecting measurement of a pulse wave has occurred, the operation control unit 1053 controls power supply circuitry (not shown) so as to cut power supply to the other units in the blood pressure monitor 1 except the processing unit 12 for a certain period of time set in advance. In addition, the operation control unit 1053, for example, controls the power supply circuitry so as to cut power supply to all the sensor units except the first sensor unit 130-1 in a period from the time when the occurrence of the body motion is detected to the time when the occurrence of the body motion is no longer detected. Therefore, it is possible to reduce wasteful power consumption caused by operating the sensor units during a period in which a body motion is occurring and measurement cannot be properly performed.

In general, if the measurement operation is always performed regardless of whether a body motion is stationary or is occurring, wasteful power consumption corresponding to the measurement time during the body motion may occur. In particular, a battery life is one of important design issues of wearable devices such as the blood pressure monitor described above. In the second embodiment, the power supply to each sensor unit and the like can be controlled according to the state of the occurrence of the body motion; therefore, an effective power-saving operation can be performed, and the battery life can be extended.

Also, the PTT calculator 103 and the blood pressure estimator 104 do not perform the processing of calculating a PTT and the processing of estimating a blood pressure value; therefore, an inaccurate blood pressure estimate value affected by the body motion is not stored in the measurement value storage 142. Therefore, the measurement accuracy of the blood pressure estimate value can be improved.

### Modifications

(1) Example of System Including Blood Pressure Monitor 1

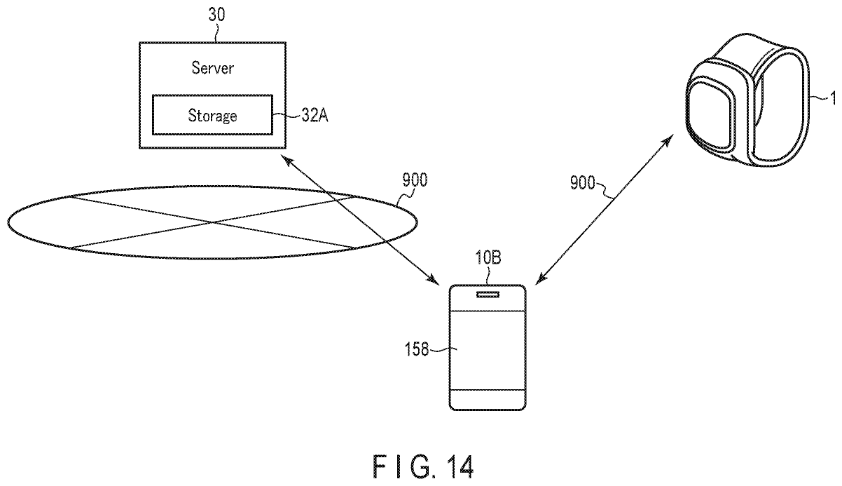

FIG. 14 is a diagram illustrating a schematic configuration of a system including the blood pressure monitor 1 described in the first and second embodiments. The blood pressure monitor 1 communicates with a server 30 or a portable terminal 10B, which is an external information processor, via a network 900. In the system shown in FIG. 14, the blood pressure monitor 1 communicates with the portable terminal 10B via the LAN, and the portable terminal 10B communicates with the server 30 via the Internet. Thus, the blood pressure monitor 1 can communicate with the server 30 via the portable terminal 10B. The blood pressure monitor 1 may communicate with the server 30 without going via the portable terminal 10B.

For example, an indication of whether or not there is a body motion while the blood pressure monitor 1 is worn, an alarm, or the like may be displayed on the display 50 of the blood pressure monitor 1, or a detection result of whether or not there is a body motion, a state of transition to the power-saving mode, or the like may be transmitted to the portable terminal 10B and displayed on a display 158. Thereby, the blood pressure monitor 1 can output the state of the occurrence of the body motion from the indication of the display 158 of the portable terminal 10B. The aforementioned indication, or the like may also be displayed on both the display 50 and the display 158 of the blood pressure monitor 1. In addition, the portable terminal 10B may report information indicating whether or not there is an occurrence of a body motion or information indicating the operation mode of the blood pressure monitor 1 in other output modes including vibration of the portable terminal 10B or a sound. The calculated blood pressure and body motion log is not necessarily stored in the measurement value storage 142 and the body motion storage 143 of the blood pressure monitor 1, but may be stored in a storage of the portable terminal 10B or a storage 32A of the server 30. Alternatively, the calculated blood pressure and body motion log may be stored in two or more of these storages.

(2) In each of the above-described embodiments, the blood pressure monitor that estimates blood pressure from a pulse wave velocity PTT using at least two pairs of pulse wave sensors 130 is described. However, the embodiments of the present disclosure may be a pulse wave measurement apparatus including only one pair of pulse wave sensors (that is, one transmission antennas and one reception antenna).

(3) In each of the above-described embodiments, the pulse wave sensor 130 using a radio wave is described. However, a pulse wave sensor using another principle such as a photoelectric method or a piezoelectric method may also be used.

(4) In each of the above-described embodiments, the case where a pulse wave is measured in the radial artery 91 of the wrist is described as an example. However, a pulse wave may be measured in other parts such as the upper arm, the ankle, and the thigh.

(5) It is also conceivable that an operation of removing the blood pressure monitor 1 from the measurement site is detected by the body motion determination unit 105, thereby automatically shifting the apparatus 1 to the power-saving mode through the removal operation or turning off the power of the apparatus 1.

(6) Several examples in which the feature of the waveform of the pulse wave signal is compared with the preset threshold are described as methods of detecting a state of an occurrence of a body motion. However, detailed determination conditions may be reversed depending on the polarity of the signal, as described above. The features of the waveform described above can also be replaced by other equivalent or complementary features. In this manner, the detailed determination conditions illustrated as examples above can be variously modified in accordance with the circuitry design, the operating environment, and the like, and are not limited to the above embodiments.

While the embodiments of the present invention have been detailed, the foregoing description is merely illustrative of the present invention in all respects. It goes without saying that various improvements and modifications can be made without departing from the scope of the present invention. For example, the following modifications can be made. In the following description, the same components as those of the above-described embodiments are denoted by the same reference numerals, and description of the same points as those of the above-described embodiments is omitted as appropriate. The following modifications can be combined as appropriate.

### APPENDIX

A part or whole of each of the above-described embodiments may be described as, but is not limited to, what is described in the appendices below, in addition to what is described in the claims.

### Appendix 1

A biological information measurement apparatus including a hardware processor and a memory,

the biological information measurement apparatus configured to
transmit a radio wave to a measurement site of a living body; and
receive a reflected wave of the radio wave by the measurement site and output a waveform signal of the reflected wave,

the hardware processor configured to, by executing a program stored in the memory,
extract information indicating a feature of a waveform from the waveform signal; and
detect a state of an occurrence of a body motion of the living body affecting measurement of the biological information, based on the extracted information indicating the feature of the waveform.

### Appendix 2

A biological information measurement method executed by an apparatus including a hardware processor and a memory storing a program for executing the hardware processor, the biological information measurement method including:

transmitting a radio wave to a measurement site of a living body;

receiving a reflected wave of the radio wave by the measurement site and outputting a waveform signal of the reflected wave; and

the hardware processor extracting information indicating a feature of a waveform from the waveform signal; and

the hardware processor detecting a state of an occurrence of a body motion of the living body affecting measurement of the biological information, based on the extracted information indicating the feature of the waveform.

### Appendix 3

A biological information measurement apparatus (1) for measuring biological information, the biological information measurement apparatus (1) including:

a transmitter (3) configured to transmit a radio wave to a measurement site of a living body;

a receiver (4) configured to receive a reflected wave of the radio wave by the measurement site and output a waveform signal of the reflected wave;

a feature extractor (1051) configured to extract information indicating a feature of a waveform from the waveform signal; and

a body motion detector (1052) configured to detect a state of an occurrence of a body motion of the living body affecting measurement of the biological information, based on the extracted information indicating the feature of the waveform.

### REFERENCE SIGNS LIST

1. Biological information measurement apparatus (blood pressure monitor)
2. Sensor unit
3. Transmitter
4. Receiver
5. Output unit
10. Main body
12. Processing unit
13. Sensing unit
14. Storage unit
16. Input/output interface
17. Communication interface
20. Strap
30. Server
40. Transmitter/receiver
50. Display
52. Controller
90. Wrist
91. Radial artery
101. Pulse wave detector
103. PTT calculator
104. Blood pressure estimator
105. Body motion determination unit
130. Sensor unit
141. Correspondence equation storage
142. Measurement value storage
143. Body motion storage
158. Display
900. Network
1051. Feature extractor
1052. Body motion detector
1053. Operation control unit

1. A biological information measurement apparatus for measuring biological information, the apparatus comprising:
a transmitter configured to transmit a radio wave to a measurement site of a living body;
a receiver configured to receive a reflected wave of the radio wave by the measurement site and output a waveform signal of the reflected wave;
a feature extractor configured to extract information indicating a feature of a waveform from the waveform signal; and
a body motion detector configured to detect a state of an occurrence of a body motion of the living body affecting measurement of the biological information, based on the extracted information indicating the feature of the waveform.

2. The biological information measurement apparatus according to claim 1, wherein
the feature extractor is configured to extract information relating to an amplitude of the waveform signal as a feature of the waveform of the waveform signal, and
the body motion detector is configured to determine that the body motion has occurred when an amplitude value of the waveform signal exceeds a preset first amplitude value for a time period longer than a preset first duration, based on the extracted information relating to the amplitude of the waveform.

3. The biological information measurement apparatus according to claim 1, wherein
the feature extractor is configured to extract information relating to an amplitude of the waveform signal as a feature of the waveform of the waveform signal, and
the body motion detector is configured to determine that the body motion has occurred when an amplitude value of the waveform signal is below a preset first amplitude value for a time period shorter than a preset first duration, based on the extracted information relating to the amplitude of the waveform.

4. The biological information measurement apparatus according to claim 1, wherein
the feature extractor is configured to extract information relating to an amplitude of the waveform signal as a feature of the waveform of the waveform signal, and
the body motion detector is configured to determine that the body motion has occurred when an amplitude value of the waveform signal exceeds a preset first amplitude value for a time period shorter than a preset first duration, based on the extracted information relating to the amplitude of the waveform.

5. The biological information measurement apparatus according to claim 1, wherein
the feature extractor is configured to extract information relating to an amplitude of the waveform signal as a feature of the waveform of the waveform signal, and
the body motion detector is configured to determine that the body motion has occurred when an amplitude value of the waveform signal is below a preset first amplitude value for a time period longer than a preset first duration, based on the extracted information relating to the amplitude of the waveform.

6. The biological information measurement apparatus according to claim 1, wherein
the feature extractor is configured to extract information relating to a repetition cycle of the waveform signal as a feature of the waveform of the waveform signal, and
the body motion detector is configured to determine that the body motion has occurred when the repetition cycle of the waveform signal exceeds a preset time range, based on the extracted information relating to the repetition cycle of the waveform.

7. The biological information measurement apparatus according to claim 1, wherein
the feature extractor is configured to extract information relating to an amplitude of the waveform signal as a feature of the waveform of the waveform signal, and
the body motion detector is configured to determine that the body motion has occurred when an amplitude value of the waveform signal exceeds a preset first amplitude range, based on the extracted information relating to the amplitude of the waveform.

8. The biological information measurement apparatus according to claim 1, wherein
the feature extractor is configured to extract information relating to an amplitude of the waveform signal as a feature of the waveform of the waveform signal, and
the body motion detector is configured to determine that the body motion has occurred when an amplitude value of the waveform signal does not exceed a preset second amplitude range, based on the extracted information relating to the amplitude of the waveform.

9. The biological information measurement apparatus according to claim 1, wherein
the feature extractor is configured to extract, as a feature of the waveform of the waveform signal, information relating to an amplitude of a waveform of the waveform signal in each repetition interval, and
the body motion detector is configured to determine that the body motion has occurred when a difference between an amplitude value of a waveform in a first repetition interval and an amplitude value of a waveform in a second repetition interval, different from the first repetition interval, exceeds a preset second amplitude range, based on the extracted information relating to the amplitude of the waveform in each repetition interval of the waveform.

10. The biological information measurement apparatus according to claim 1, wherein
the feature extractor is configured to extract, as a feature of the waveform of the waveform signal, information relating to a spectrum intensity of a predetermined frequency band for each preset time interval of the waveform signal, and
the body motion detector is configured to determine that the body motion has occurred when the information relating to the spectrum intensity exceeds a preset range, based on the extracted information relating to the spectrum intensity.

11. The biological information measurement apparatus according to claim 1, wherein
the feature extractor is configured to extract, as a feature of the waveform of the waveform signal, information indicating a shape of a waveform of the waveform signal in each repetition interval, and
the body motion detector is configured to determine that the body motion has occurred when a correlation value between the shape of the waveform extracted and a shape of a reference waveform stored in advance is equal to or less than a preset correlation value, based on the extracted information relating to the shape of the waveform.

12. The biological information measurement apparatus according to claim 1, wherein
the feature extractor is configured to extract, as a feature of the waveform of the waveform signal, information indicating a shape of a waveform of the waveform signal in each repetition interval, and
the body motion detector is configured to determine that the body motion has occurred when a correlation value between a shape of a waveform in a first repetition interval and a shape of a waveform in a second repetition interval different from the first repetition interval is equal to or less than a preset correlation value, based on the extracted information relating to the shape of the waveform.

13. The biological information measurement apparatus according to claim 1 wherein the body motion detector is configured to periodically perform an operation of determining an occurrence of the body motion, and return to the operation of determining an occurrence of the body motion when it is not determined that the body motion has occurred continuously for a preset period of time, or when it is not determined that the body motion has occurred continuously for a preset number of cycles after the occurrence of the body motion is determined.

14. The biological information measurement apparatus according to claim 1, wherein the body motion detector further comprises an operation control unit configured to stop power supply to at least one of the transmitter, the receiver, the feature extractor, or the body motion detector for a preset time period when the occurrence of the body motion is detected.

15. The biological information measurement apparatus according to claim 13, wherein the body motion detector further comprises an operation control unit configured to stop power supply to at least one of the transmitter, the receiver, the feature extractor, or the body motion detector from a time point when the occurrence of the body motion is detected to a time point when the body motion detector returns to the operation of determining an occurrence of the body motion.

16. The biological information measurement apparatus according to claim 1, further comprising an output unit configured to output a result of detection by the body motion detector.

17. A biological information measurement method performed by a biological information measurement apparatus for measuring biological information, the method comprising:
transmitting a radio wave to a measurement site of a living body;
receiving a reflected wave of the radio wave by the measurement site and outputting a waveform signal of the reflected wave;
extracting information indicating a feature of a waveform from the waveform signal; and
detecting a state of an occurrence of a body motion of the living body affecting measurement of the biological information, based on the extracted information indicating the feature of the waveform.

18. A non-transitory computer readable medium storing a computer program for causing a computer to execute the steps of:
transmitting a radio wave to a measurement site of a living body;
receiving a reflected wave of the radio wave by the measurement site and outputting a waveform signal of the reflected wave;
extracting information indicating a feature of a waveform from the waveform signal; and
detecting a state of an occurrence of a body motion of the living body affecting measurement of the biological information, based on the extracted information indicating the feature of the waveform.
